SEQUENCE LISTING (4 sequences)

SEQ ID NO: 1 - DNA, 20 bp, Artificial Sequence
  The sequence is synthetized.
ccagacagcagccaattgtc

SEQ ID NO: 2 - DNA, 20 bp, Artificial Sequence
  The sequence is synthetized.
ggtagaagaccccctcttac

SEQ ID NO: 3 - DNA, 20 bp, Artificial Sequence
  The sequence is synthetized.
ctccatcctggcctcgctgt

SEQ ID NO: 4 - DNA, 20 bp, Artificial Sequence
  The sequence is synthetized.
gctgtcaccttcaccgttcc

(19) United States
(12) Patent Application Publication
(10) Pub. No.: US 2023/0346935 A1
(43) Pub. Date: Nov. 2, 2023

(54) TUMOR COMPLEX ANTIGEN, MULTIVALENT DENDRITIC CELL (DC) VACCINE, AND USE THEREOF

(71) Applicants: Helen LIU, Shanghai (CN); KOUSAI Bio Co., Ltd, Shanghai (CN)

(72) Inventors: Helen LIU, Shanghai (CN); Ze YIN, Shanghai (CN)

(73) Assignees: Helen LIU, Shanghai (CN); KOUSAI Bio Co., Ltd, Shanghai (CN)

(21) Appl. No.: 18/246,194

(22) PCT Filed: Aug. 4, 2021
(86) PCT No.: PCT/CN2021/110455

§ 371 Date: Mar. 22, 2023

(30) Foreign Application Priority Data
Sep. 24, 2020 (CN) 202011017224.5

(51) Int. Cl. A61K 39/00; A61K 39/39; A61P 35/00

(52) U.S. Cl. CPC A61K 39/4615; A61K 39/4622; A61K 39/464499; A61K 39/39; A61P 35/00; A61K 2039/70; A61K 2039/5154; A61K 2039/55522; A61K 2039/55538; A61K 2039/55527

(57) ABSTRACT

A tumor complex antigen, a multivalent dendritic cell (DC) vaccine, and a use thereof are provided. In the present disclosure, monocytes of a patient are stimulated in vitro, loaded with a variety of tumor cell lysates with strong immunogenicity against different Epstein-Barr virus (EBV)-associated tumors, and induced into mature dendritic cells (mDCs) by various cytokines and specific agonists to obtain a complete DC vaccine with corresponding tumor antigens. The DC vaccine can be injected back into the patient to activate an immune system, stimulate innate immunity (such as inducing natural killer (NK) cells), and stimulate lymphocytes to produce an acquired immune response and cytotoxic T cells, thereby accurately killing tumor cells. Compared with radiotherapy and chemotherapy, the DC vaccine is particularly safe and has almost no side effects. In addition, the production of the DC vaccine involves a short production cycle of about 1 week and a low cost.

### CROSS REFERENCE TO THE RELATED APPLICATIONS

This application is the national phase entry of International Application No. PCT/CN2021/110455, filed on Aug. 4, 2021, which is based upon and claims priority to Chinese Patent Application No. 202011017224.5, filed on Sep. 24, 2020, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBSHSS001_Sequence_Listing.txt, created on Sep. 23, 2022, and is 1,132 bytes in size.

### TECHNICAL FIELD

The present disclosure relates to the technical field of biomedicine, and in particular to a tumor complex antigen, a multivalent dendritic cell (DC) vaccine, and a use thereof.

### BACKGROUND

Epstein-Barr virus (EBV) is a member of the lymphotropic virus genus of the herpesvirus family, which has a DNA genome with a length of about 170 kb. People are generally susceptible to EBV, and EBV spreads easily with an infection rate as high as 95% among adults. EBV can be carried for life, and there are regional differences in diseases caused by EBV. EBV infection mostly occurs in the childhood and adolescence. EBV infection in the body may develop into long-term latent infection in some cases and may also lead to various human malignant tumors, such as Burkitt's lymphoma (BL), nasopharyngeal carcinoma (NPC), Hodgkin's lymphoma (HL), non-Hodgkin's lymphoma (NHL), gastric carcinoma (GC), breast cancer, and tumors in immunodeficiency patients. In addition, EBV can specifically infect human and some primate B cells in vivo or in vitro and can stimulate the continuous growth of infected cells and cause the unlimited passage of cells to achieve immortalization, thereby producing lymphoblastoid cell lines (LCLs). Such cell lines are often used to study the occurrence and development of various diseases, which makes it possible to study the pathogenesis of some diseases on a large scale for a long time. However, the pathogenesis of EBV is still not fully understood, and the research and treatment for EBV-associated tumors (such as EBV-associated GC and NPC with high incidence in Southern China) still need to be improved.

GC is a malignant tumor originating from the gastric mucosa epithelium and is one of the most common tumors worldwide. GC is common in people aged 50 or older, but there is an upward trend in younger populations being diagnosed with GC. According to statistics of the International Agency for Research on Cancer (IARC), in 2012, there were about 951,000 new GC cases and about 723,000 GC deaths worldwide, where the incidence ranked fifth and the mortality ranked third among malignant tumors. More than 70% of the new GC cases occurred in developing countries, and about 50% of the new GC cases occurred in East Asia, mainly in China. In 2012, the new GC cases and the GC deaths in China accounted for 42.6% and 45.0% of the global new GC cases and the global GC deaths, respectively. In 2015, there were approximately 679,000 new GC cases and about 498,000 GC deaths in China. The GC incidences in the northwest and eastern coastal regions of China were significantly higher than the GC incidence in the southern region, causing extremely heavy disease burden. The specific pathogenesis of GC is complex and has not yet been fully understood, but a large number of research data show that the occurrence of GC is a result of the combined action of various factors, such as geographical environment, dietary and living factors, Helicobacter pylori (H. pylori) infection, EBV infection, precancerous lesions, inheritance, and gene mutation. Studies have reported that EBV-associated GCs account for 10% of all GCs. GC can occur in any part of the stomach, but more than half of GCs occur in the gastric antrum although GCs may occur in the greater curvature of stomach, the lesser curvature of stomach, and the anterior and posterior walls as well. The vast majority of GCs are adenocarcinomas and have no obvious symptoms or have non-specific symptoms, such as epigastric discomfort and belching in an early stage, which are often similar to symptoms of chronic gastric diseases, such as gastritis and gastric ulcer, and thus are easily ignored. Therefore, an early diagnosis rate of GC in China is currently low, and it is urgent to discover a GC-specific marker and improve the early diagnosis rate of GC. In addition, the treatment of GC has shown a trend of multidisciplinary treatment. The combined use of surgery, radiotherapy, targeted therapy, and immunotherapy may help the treatment and prognosis of GC and the pain relief of a patient.

Among EBV-associated tumors, NPC is a malignant tumor occurring on an epithelium of the nasopharynx and is almost a squamous cell carcinoma (SCC). NPC progresses rapidly, which is highly invasive and metastatic and is accompanied by headache, tinnitus, and hearing or vision loss. Epidemiologically, the incidence of NPC is very regional and is high in Southeast Asia, the Middle East, Southern Africa, and Southern China. In particular, an incidence of NPC in Guangdong province of China is as high as 30/100,000, and NPC is one of the most common malignant tumors in Southern China. According to the World Health Organization (WHO) in 2012, there are about 33,000 new NPC cases and 20,000 NPC deaths in China every year. At present, a diagnosis method of NPC mainly relies on tissue biopsy by a nasal endoscope, which is invasive to some extent. Moreover, due to the lack of serological diagnostic markers with both high sensitivity and high specificity, it is difficult for NPC to become a routine examination item in the hospital. At present, due to the special location of NPC, the early detection of NPC is difficult in clinical practice, and surgical resection is not suitable. With the progression of NPC, a 5-year survival rate of NPC patients declines rapidly. For example, a 5-year survival rate of NPC patients at an early clinical stage is 90%, while a 5-year survival rate of NPC patients at an advanced clinical stage IV is less than 50%. In addition, after radiotherapy and chemotherapy, NPC easily relapses and metastasizes, and the radiotherapy and chemotherapy have large side effects. Therefore, if not detected and treated early, NPC will pose a great threat to the life of a patient. The development of a safe and effective treatment for relapsed and advanced stage NPC is an urgent need for NPC patients.

Currently, the pathogenesis of NPC is still uncertain. Some studies have pointed out that the incidence of NPC is mainly related to genetic factors, environmental regionality, dietary habits (pickled foods), and EBV infection, among which EBV has been confirmed to be highly correlated with the occurrence and development of NPC. At present, NPC is mainly treated by conventional radiotherapy and chemotherapy, which can achieve some therapeutic effects (normally, a 3-year survival rate is 80% after treatment) but will bring much discomfort to a patient during the treatment; and there is no specific drug for NPC. Therefore, the development of preventive and therapeutic NPC vaccines has always been a research hotspot. In addition, due to great differences among different NPC patients, biological processes in cancer cells of different patients are different, and expression levels of various proteins are also different. Therefore, the effects of currently known markers or specific drugs are often not favorable in all patients. Therefore, clinically, there is an urgent need for a reagent or vaccine with high specificity that is directly derived from a patient and can be used for the diagnosis and treatment of NPC.

The research on NPC vaccines for many years has mainly focused on preventive vaccines with EBV gp350 as a target antigen and single or combined recombinant therapeutic vaccines with EBV-EBNA1, LMP1, and LMP2 as target sites. At present, some substantial progress has been made in the studies of relevant vaccines in China, and the phase I and II clinical trials have been conducted in many of the studies. However, there is no specific tumor marker for the occurrence and development of NPC, making it difficult to conduct subsequent clinical trials. The current research models for the related vaccines are mostly mice, rhesus monkeys, cynomolgus monkeys, and the like, and thus the scientific research basis is insufficient.

In addition, as a B-cell-tropic human herpesvirus, EBV can latently reside in infected B cells for a long time to cause persistent latent infection, which is closely related to the occurrence, development, and prognosis of various lymphomas, including BL, diffuse large B-cell lymphoma (DLBCL), HL, NK/T-cell lymphomas, and the like. The WHO estimated that there were 589,600 new lymphoma cases worldwide in 2018, including 80,000 HL cases and 509,600 NHL cases with a global standardized incidence of 6.6/100,000. In China, a detection rate of EBV in HL patients can reach 48% to 57%. The treatment of EBV-associated lymphomas is complicated. In addition to the selection of sensitive chemotherapy and traditional local radiotherapy according to different lymphoma types, it is necessary to design a treatment plan based on the characteristics of EBV to effectively clear EBV infection and activity from the body, attenuate a driving action of EBV for disease progression, and block the further development of lymphomas. A supplementary EBV-specific treatment based on a traditional treatment method, such as antiviral therapy and immunotherapy, is essential to improve the efficacy for EBV-associated lymphomas.

Some studies have shown that DCs in EBV-associated tumor patients have a low degree of differentiation, a reduced quantity, an impaired ability to recognize and present antigens, and a weak ability to activate naive T cells, which ultimately makes the body fail to recognize and clear tumor cells. EBV infection is very likely one of the reasons for impaired DC functions in patients with NPC and other related cancers. DCs were discovered in 1973 by the Canadian scientist Ralph M. Steinman, winner of the 2011 Nobel Prize in Medicine and Physiology. DCs are named for their dendritic or pseudopod-like protrusions that protrude in a mature stage. DCs are the most powerful professional antigen-presenting cells (APCs) currently known in the body, which can efficiently take up, process, and present antigens. DCs are the only APCs found so far that can activate unsensitized naive T cells. In addition, immature dendritic cells (iDCs) have a strong ability to transfer and take up antigens, and mature dendritic cells (mDCs) can effectively activate naive T cells and are in a central link for initiating, regulating, and maintaining an immune response. A quantity of DCs is less than 1% of a quantity of peripheral blood mononuclear cells (PBMCs). The surface of a DC is rich in antigen-presenting molecules (such as MHC-I and MHC-II), costimulatory factors (such as CD80/B7-1, CD86/B7-2, and CD40), and adhesion factors (such as ICAM-1, ICAM-2, ICAM-3, LFA-1, and LFA-3). Therefore, DCs are an important group of innate immune cells and professional APCs and play a key regulatory role in the activation of an immune response in the body and in the maintenance of autoimmune tolerance.

As the strongest APCs, DCs can effectively present antigen information to T cells and induce the activation of T cells, thereby causing a series of immune responses. An MHC molecule on the surface of DCs can bind to an antigen to produce a peptide-MHC molecular complex, thereby presenting an antigen signal to T cells. Some costimulatory factors (such as CD80/B7-1, CD86/B7-2, and CD40) highly expressed on DCs provide a second signal necessary for the activation of T cells. DCs can also directly present an antigenic peptide to CD8⁺ T cells and activate CD8⁺ T cells with the help of CD4⁺ T cells. Activated DCs can secrete IL-12, IL-18, chemotactic cytokines (CCK), and the like in large quantities to activate the proliferation of T cells and initiate an MHC-class I-restricted cytotoxic T lymphocyte (CTL) response and an MHC-class II-restricted CD4⁺ Th1 immune response. In addition, DCs can also activate perforin-dependent and FasL/Fas-mediated pathways to enhance NK cytotoxicity, thereby enhancing the body's anti-tumor immune response to eliminate tumors. DCs themselves can serve as a natural immune adjuvant to improve the immune competence of the body by secreting various cytokines and can also enhance immune responses of various vaccines. Generally, DCs with relevant antigen information and vaccine functions are called DC vaccine.

Vaccines are preventive or therapeutic biological products for human vaccination, which play an important role in preventing, treating, and controlling the occurrence and prevalence of infectious diseases. However, with the increasing production and use of vaccines, problems such as excessive inoculation doses and limited therapeutic effects also arise. For example, the 14 vaccines in the routine immunization of children in the immunization plan of China require a total of 17 to 19 injections before the age of 3 (17 injections: the Japanese encephalitis virus (JEV) live attenuated vaccine and the hepatitis A live attenuated vaccine are used to complete immunization; and 19 injections: the JEV inactivated vaccine and the hepatitis A inactivated vaccine are used to complete immunization). The large number of immunization injections is inconvenient to parents and medical staff, may decrease all-course vaccination rate, and increase the cost of immunizations due to immunization time inconsistance. There may also be an increase in the likelihood of adverse reactions from immunization, which increases the difficulty in supervision and clinical use of vaccines. In order to reduce the number of immunization injections, improve the prevention and treatment effects, reduce the implementation cost of the expanded programme on immunization (EPI), and maximize the disease prevention and treatment effects of vaccines, multivalent vaccines or combined vaccines must be developed and promoted, which is particularly important in reducing the implementation cost and maximizing the disease treatment effect.

A vaccine with only a single antigenic component is called monovalent vaccine, and a monovalent vaccine can only prevent one infectious disease or one type of pathogen infection. A vaccine prepared by mixing two or more antigenic components in an appropriate ratio is called multivalent vaccine or combined vaccine. For example, there are more than 100 types of human papillomavirus (HPV), most of which only cause skin warts. Some types of HPV can cause cervical cancer, such as HPV 16, 18, 31, 33, 35, 39, 45, 51, 52, 56, 58, 59, 68, 69, 73, and 82, which are called high-risk HPV; other types of HPV are low-risk HPV. A bivalent HPV vaccine can only prevent the infection of HPV 16 and 18 (high-risk HPV), and 70% of cervical cancers are caused by these two types of HPV. A quadrivalent HPV vaccine can prevent the infection of HPV 16, 18, 6, and 11, among which the HPV 6 and 11 are low-risk HPV and are the main causes of genital warts. A nine-valent HPV vaccine can prevent the infection of HPV 16, 18, 31, 33, 45, 52, 58, 6, and 11 (namely, 7 high-risk HPV types and 2 low-risk HPV types) and is currently a vaccine that can target the most types of HPV. The development of multivalent vaccines has a history of nearly a hundred years, and the research on combined vaccines began as early as the 1930s. In 1945, the trivalent influenza vaccine was approved for use in the United States for the first time, and then the hexavalent pneumococcal vaccine, the Diphtheria-Tetanus (DT) double vaccine, the Diphtheria-Pertussis-Tetanus (DPT) triple vaccine, and the trivalent oral poliovirus attenuated live vaccine were successively developed. Results of clinical trials have shown that the combined immunization with a multivalent vaccine is often superior to the multi-vaccination with a monovalent vaccine. When a multivalent vaccine is used for combined immunization, an immunization effect is similar to or better than an immunization effect of a monovalent vaccine, and the side effects of the vaccine are not increased.

Defects and deficiencies of the prior art: Due to difficult early detection of EBV-associated tumors such as NPC and GC, these tumors are often found in middle and late stages and thus are mainly treated by traditional methods. The traditional surgery, radiotherapy, and chemotherapy exhibit an inhibition rate of about 20% for tumor cells and an inhibition rate of about 90% for lymphocytes, and excessive chemotherapy will decrease the likelihood of the patient's survival. Some tumors are not susceptible to drugs, and chemotherapy has no clinical value for such tumors. Some tumors often shrink rapidly after radiotherapy and chemotherapy, but because tumor cells are not eliminated, the tumors will grow rapidly and have extensive proximal and distant metastasis, which accelerates the metastasis of tumor cells during treatment and destroys the immune system. Therefore, some patients die not from cancer but from complications after overtreatment. Currently, there is an urgent need to find new drugs or methods for treating cancer in clinical. Compared with traditional treatment methods, immunotherapy has gradually become a new cancer treatment method due to its prominent therapeutic effect and few side effects. DC vaccines are playing an increasingly important role in cancer treatment and prevention, but monovalent DC vaccines often lead to a limited therapeutic effect. Different antigen types and combinations can be used to treat various diseases. However, since most tumor cells are derived from the human body, tumor cells can easily evade treatments based on single tumor antigen, so that the immune response from the treatment does not successfully kill a tumor, resulting in limited therapeutic effects. In addition, because many tumor cells in vivo can secrete a variety of cytokines that inhibit the maturation of DCs, there is relatively a small number of DCs at a tumor site, and an anti-tumor immune response induced by DCs in the absence of strong tumor antigen stimulation cannot lead to a very significant therapeutic effect in a host.

### SUMMARY

In view of the deficiencies of the prior art, the present disclosure is intended to provide a tumor complex antigen, a multivalent DC vaccine, and a use thereof. In the present disclosure, DCs of a patient are stimulated in vitro, loaded with a variety of tumor cell lysates with strong immunogenicity against different EBV-associated tumors (e.g., a tumor cell lysate of human immortalized B lymphoblastoid cell lines (B-LCLs) derived from different EBV strains, such as SNU-719, YCCEL1, GD1, B95-8, M81, and HKNPC1 to HKNPC9, or an EBV-positive tumor cell lysate, such as C666-1, HNE1, or CCL85), and induced into mDCs by various cytokines and specific agonists to obtain a complete DC vaccine with corresponding tumor antigens. The DC vaccine can be injected back into the patient to activate an immune system, stimulate innate immunity (such as inducing natural killer (NK) cells), and stimulate lymphocytes to produce an acquired immune response and produce cytotoxic T cells, thereby accurately killing EBV-associated tumor cells to achieve personalized treatment. Compared with radiotherapy and chemotherapy, the DC vaccine is particularly safe and has almost no side effects. In addition, the production of the DC vaccine involves a short production cycle of about 1 week and a low cost.

In order to achieve the above objective, the present disclosure adopts the following technical solutions:

In a first aspect, the present disclosure provides a tumor complex antigen, including a tumor cell lysate of human immortalized B-LCLs derived from different EBV strains and/or an EBV-positive tumor cell lysate. The tumor cell lysate of human immortalized B-LCLs derived from different EBV strains is any one or a combination of two or more selected from the group consisting of GD1, B95-8, M81, HKNPC1 to HKNPC9, SNU-719, and YCCEL1. The EBV-positive tumor cell lysate is any one or a combination of two or more selected from the group consisting of C666-1, HNE1, and CCL85.

In a second aspect, the present disclosure provides a multivalent DC vaccine, which carries the tumor complex antigen described above. The multivalent DC vaccine carrying the tumor complex antigen is loaded with one, two, three, or more EBV-associated tumor cell lysates or LCL tumor cell lysates.

As a preferred solution, the tumor cell lysate of human immortalized B-LCLs derived from different EBV strains may be a tumor cell lysate of one or more selected from the group consisting of human immortalized B-LCLs resulting from transformation by EBVs of GD1, B95-8, M81, HKNPC1 to HKNPC9, SNU-719, and YCCEL1. The EBV-positive tumor cell lysate may be C666-1, HNE1, or CCL85.

Further, the multivalent DC vaccine may include a first adjuvant or a cytokine for adjuvant therapy.

Furthermore, the first adjuvant may be any one selected from the group consisting of PloyI:C, LPS, and OK432, and the cytokine for adjuvant therapy may be TNF-α or IL-12.

Furthermore, each of the tumor cell lysates may be specifically used at an amount of 2.5×10⁷ to 2.5×10⁹ cells.

In a third aspect, the present disclosure provides a use of the tumor complex antigen described above in the preparation of a drug for preventing or treating an EBV-associated tumor.

As a preferred solution, the EBV-associated tumor may include EBV-associated GC, EBV-positive lymphoma, NPC, and other EBV-associated epithelial tumors.

Further, the drug may include the multivalent DC vaccine described above.

The multivalent DC vaccine provided by the present disclosure can stimulate an immune response of the body to treat an EBV-associated tumor, especially EBV-associated GC, EBV-positive lymphoma, NPC, and other EBV-associated epithelial tumors with prominent efficacy and small side effects. The multivalent DC vaccine can effectively inhibit the growth of an EBV-associated tumor and slow down the progression of the tumor for a long time and can even achieve a complete remission state.

In some embodiments, an antigen-sensitized DC population belongs to an immunogenic composition and is loaded with a corresponding antigen. Specific antigens may include a tumor cell lysate of human immortalized B-LCLs derived from different EBV strains, such as GD1, B95-8, M81, HKNPC1 to HKNPC9, SNU-719, and YCCEL1 and a tumor cell lysate of C666-1, HNE1, or CCL85. Each of the tumor cell lysates may be specifically used at an amount of 2.5×10⁷ to 2.5×10⁹ cells. In other aspects of the present disclosure, the DC vaccine carrying the tumor complex antigen may be a multivalent DC vaccine loaded with only two EBV-associated tumor cell lysates or LCL tumor cell lysates or a multivalent DC vaccine loaded with three or more EBV-associated tumor cell lysates or LCL tumor cell lysates.

In some aspects, the multivalent DC vaccine may include a first adjuvant (such as Ploy(I:C), LPS, and OK432) or a cytokine for adjuvant therapy (such as TNF-α or IL-12). The multivalent DC vaccine may be administered intravenously, intradermally, intratumorally, intramuscularly, intraperitoneally, intranodally, subcutaneously, or topically 3 to 30 times, at an interval of one or two weeks. The multivalent DC vaccine may be injected each time at an amount of 5×10⁶ to 5×10⁸ cells.

The present disclosure has the following advantages and beneficial effects:

The multivalent DC vaccine is a vaccine that fights against a tumor by activating an immune system of a patient. Compared with the traditional treatment for EBV-associated tumors, the multivalent DC vaccine has the following advantages:
1. Surgery, chemotherapy, or radiotherapy will greatly harm the body of a patient while killing cancer cells. Due to the tumor heterogeneity of patients, most anticancer drugs, especially the new generation of targeted drugs, are only effective for a small number of patients. Since cancer cells evolve rapidly, drug resistance is easily developed, resulting in a high cancer recurrence rate. Compared with traditional chemotherapy or targeted therapy, the multivalent DC vaccine is a potential treatment of tumors. The multivalent DC vaccine directly targets immune cells in vivo and kills cancer cells by activating an immune system in vivo, which does not cause direct damage, but strengthens the immune system instead. The multivalent DC vaccine can inhibit the evolution of cancer cells, leads to a low recurrence rate, and has significantly less side effects than traditional chemotherapy and multi-targeted drugs as a whole. For example, the multivalent DC vaccine works by activating an immune system, and thus merely has the most common side effects of clinical grade I/II adverse reactions, such as fever, fatigue, dizziness, systemic muscle pain, drowsiness, which can be treated symptomatically. Therefore, the multivalent DC vaccine has a promising clinical application prospect.
2. The selection of the multivalent DC vaccine for an antigen is effective and safe for the treatment of EBV-associated tumors. Currently, in most of the international projects for treating EBV-associated tumors with a DC vaccine, a specific polypeptide fragment is adopted as an antigen. The multivalent DC vaccine of the present disclosure has a variety of tumor cell lysates, such that all antigens for activating immune responses against cancer cells are included and the actual tumor antigen information in the human body is loaded to the maximum extent, which enhances the diversity of antigens presented by DCs, maximizes the activation of human immune functions, and can induce a strong T cell response, thereby enhancing a therapeutic effect of the product.
3. The tumor cell lysates used for the multivalent DC vaccine are derived from stable tumor cell lines and are universal antigens, such that the time-consuming and labor-intensive screening of tumor antigens of each patient is not required. There is no need to worry about the binding of a patient's human leukocyte antigens (HLAs) to antigenic peptides, that is, there is no HLA restriction. Such antigens can be easily prepared with a simple process, and the tumor cell lysates can have uniform quality, which avoids the complicated antigen screening process and brings outstanding advantages in cost reduction and time conservation.
4. The multivalent DC vaccine has a long-lasting therapeutic effect and can effectively inhibit the recurrence and metastasis of an EBV-associated tumor for a long time. After being injected into a patient, the multivalent DC vaccine can make the patient produce a large number of memory T lymphocytes carrying abundant tumor antigen information, which can exist for several years to several decades. When encountering corresponding stimulation once again, the memory T lymphocytes can be rapidly activated to kill tumor cells, which can effectively prevent the recurrence and metastasis of a tumor.
5. The various universal tumor antigens used for the multivalent DC vaccine are suitable for most EBV-associated tumor patients carrying EBV-specific antigen information. Because there is no need to screen tumor antigens of each patient, the whole preparation cycle is only 1 week, which saves several months compared with the preparation of a neoantigen DC vaccine and is of great significance for cancer patients, especially advanced cancer patients. In addition, the preparation of the universal antigens can be easily standardized, which greatly reduces the costs of production and quality control.

### BRIEF DESCRIPTION OF THE DRAWINGS

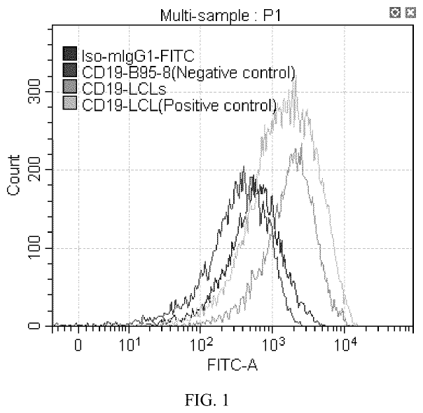

FIG. 1 shows the expression levels of CD19 on a surface of immortalized human B-LCLs,
where flow cytometry (FCM) is used to detect the expression of FITC-CD19 on a surface of B95-8 cells (negative control, CD19-B95-8), LCL-Positive control cells (positive control, CD19-LCL), and constructed EBV-transformed LCL cells (CD19-LCL). It can be known that an expression level of CD19 on a surface of CD19-LCL cells is higher than that of the isotype control (Iso-mIgG1-FITC) and CD19-B95-8 (negative control) and is comparable to that of the positive control CD19-LCL, indicating that the new LCL is successfully constructed and can be used for subsequent experiments.

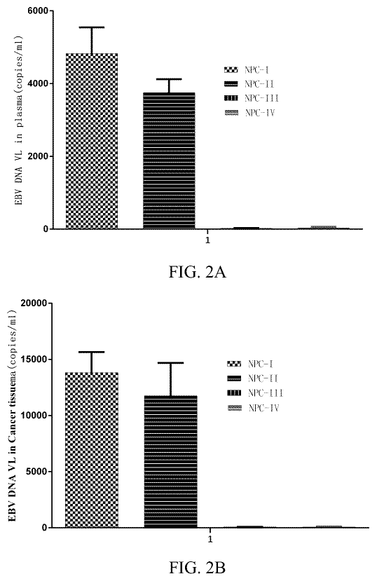

FIGS. 2A-2B show the EBV loads in the plasma and cancer tissue of 4 NPC patients,
where viral DNA is extracted from the plasma and NPC tissue of the 4 NPC patients and subjected to reverse transcription-quantitative polymerase chain reaction (RT-qPCR) with an EBV-specific test kit, and then EBV loads in the patients were calculated. It can be known that there is a large amount of EBV in the plasma and NPC tissue of EBV-positive NPC patients I and II, and a copy number of EBV-DNA in the NPC tissue is much higher than that in the plasma. EBV is basically undetectable in EBV-negative NPC patients III and IV, indicating the vigorous activities of EBV in the EBV-positive NPC patients.

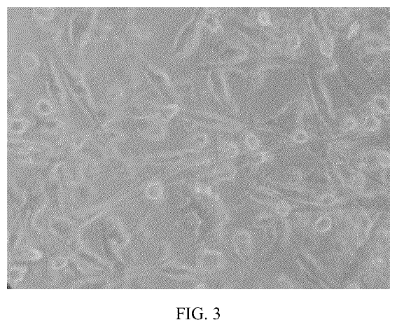

FIG. 3 shows the morphology of mDCs,
where a culture dish of mDCs is taken and placed under an optical microscope (20× objective lens) for observation. It can be seen that the mDCs grow adherently and have increased thick and long protrusions radially distributed on their surface, indicating an obvious dendritic shape.

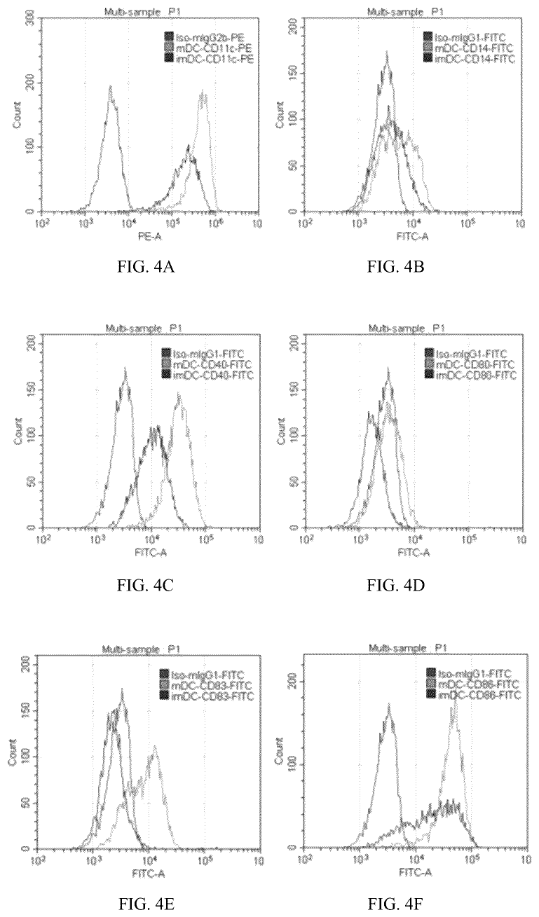

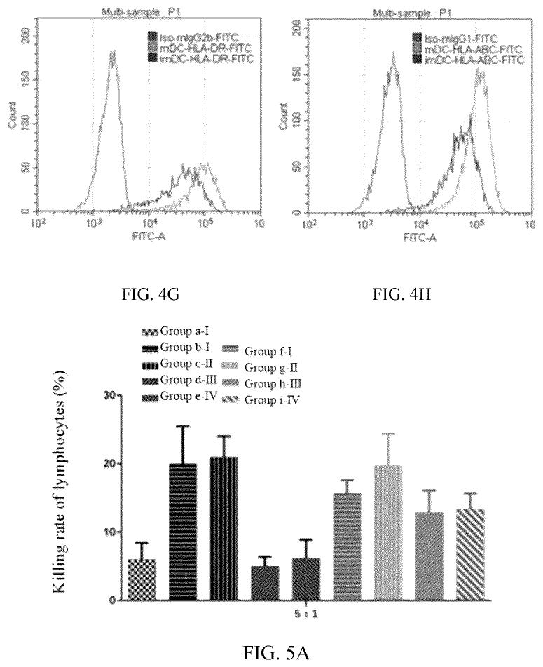

FIGS. 4A-4H show the FCM results of expression levels of marker molecules on a surface of DCs,
where some iDCs and mDCs are taken and subjected to FCM assay to determine the expression levels of cell surface factors, such as PE-CD11c/FITC-CD14/FITC-CD40/FITC-CD80/FITC-CD83/FITC-CD86/FITC-HLA-DR/FITC-HLA-ABC. It can be seen that the expression levels of CD11c, CD40, CD83, CD86, HLA-DR, and HLA-ABC on the surface of the mDCs are significantly higher than that on the surface of the iDCs, indicating that DCs are induced to be mature.

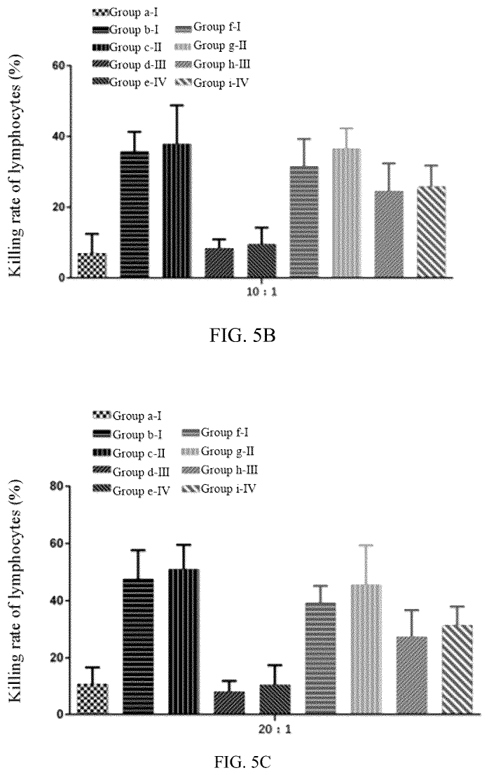

FIGS. 5A-5C show the killing rates of cytotoxic T lymphocytes (CTLs) induced by in vitro stimulation,
where in in vitro experiments, T lymphocytes stimulated by a multivalent DC vaccine, T lymphocytes stimulated only by a monovalent DC vaccine loaded with an autologous tumor cell lysate, and control cells (groups a-I) are collected to observe their ability to kill NPC cells in NPC patients. It can be seen from a comparison of killing activity that the T lymphocytes stimulated by the multivalent DC vaccine exhibits a strong killing ability for tumor cells derived from EBV-positive NPC patients (patients I and II), and the more the T lymphocytes, the more significant the killing effect. The T lymphocytes stimulated by the multivalent DC vaccine cannot effectively kill tumor cells derived from EBV-negative NPC patients (patients III and IV). CTLs stimulated by monovalent DC vaccines derived from different patients all exhibit a significant effect of killing autologous tumor cells, which is comparable to a killing effect of lymphocytes stimulated by the multivalent DC vaccine for EBV-positive NPC cells. It is evident that the DCs loaded with a variety of EBV-positive tumor cell lysates can lead to the same tumor-killing effect as a monovalent DC vaccine loaded with an autologous tumor cell lysate, which avoids the tedious process of collecting a tumor tissue from each EBV-positive NPC patient and can widely recognize EBV-positive NPC cells, stimulate the proliferation of T cells, and exert an immunological effect.

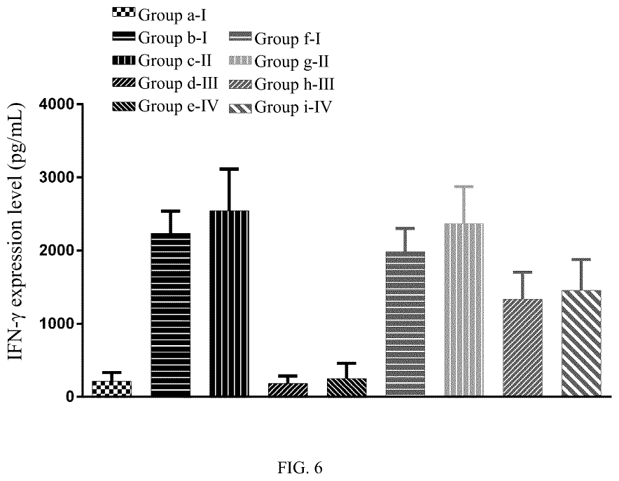

FIG. 6 shows the secretion of interferon-gamma (IFN-γ),
where CTLs in each group are co-cultivated with tumor tissue cells of an NPC patient at an effector-target ratio of 20:1, and an amount of IFN-γ secreted by the lymphocytes is detected. Based on the detection results, T lymphocytes of 4 NPC patients stimulated by the monovalent DC vaccine loaded with an autologous tumor cell lysate can produce a large amount of IFN-γ after being co-cultivated with tumor cells, and the IFN-γ level is significantly higher than that of the control group. The multivalent DC vaccine can also strongly stimulate and activate T cells in patients I and II to produce a large amount of IFN-γ, and T cells in the two EBV-negative NPC patients III and IV stimulated by the multivalent DC vaccine cannot effectively produce IFN-γ when co-cultivated with their own EBV-negative NPC cells. The above results indicate that the multivalent DC vaccine loaded with EBV-positive tumor cell lysates can strongly promote the differentiation of autologous T lymphocytes in EBV-positive NPC patients, the secretion of IFN-γ, and the anti-tumor ability of the body.

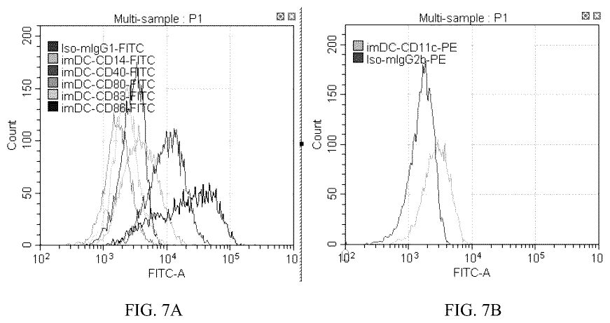

FIGS. 7A-7B show the expression of costimulatory factors on a surface of iDCs,
where the expression of costimulatory factors on the surface of iDCs is detected by FCM. CD11c is weakly expressed with a higher expression level than the control, CD80 and CD83 are not expressed, CD40 and CD86 are highly expressed to varying degrees, and the surface specific marker CD14 for monocytes is not detected, indicating that CD14 monocytes are successfully differentiated into iDCs.

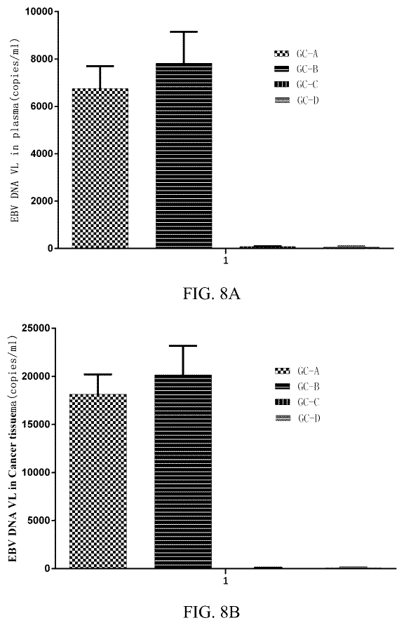

FIGS. 8A-8B show the EBV loads in plasma of 4 GC patients,
where viral DNA is extracted from the cancer tissue and plasma of the 4 GC patients and subjected to RT-qPCR with an EBV-specific test kit, and an EBV load at a corresponding site of a patient is calculated. Based on calculations, in EBV-positive GC patients A and B, an EBV load in the plasma is more than 6,000 copies, and an EBV load in the cancer tissue is more than 17,000 copies; EBV is basically undetectable in the plasma of EBV-negative GC patients C and D, indicating that there is a large amount of EBV in the EBV-positive GC patients.

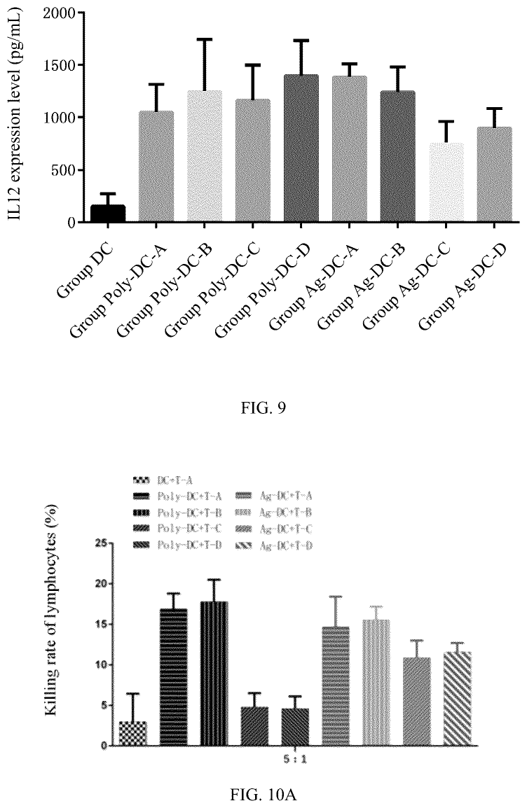

FIG. 9 shows the expression of IL12 in a culture supernatant of each vaccine group,
where an expression level of IL12 in a culture supernatant of each DC vaccine group is detected by enzyme-linked immunosorbent assay (ELISA). It is found that expression levels of IL12 stimulated by multivalent vaccines and monovalent vaccines derived from different patients are significantly higher than an expression level of IL12 secreted by normal mDCs in the control group. There is little difference in the IL12 expression level among the multivalent vaccines or monovalent vaccines derived from different patients, indicating that the GC tumor cell lysate and the GC-associated tumor antigen both can stimulate DCs to secrete IL12. The multivalent vaccine loaded with tumor antigen information of various EBV-positive GCs has the same ability as monovalent vaccines loaded with autologous tumor cell lysates of the patients to stimulate the maturation and IL12 secretion of DCs.

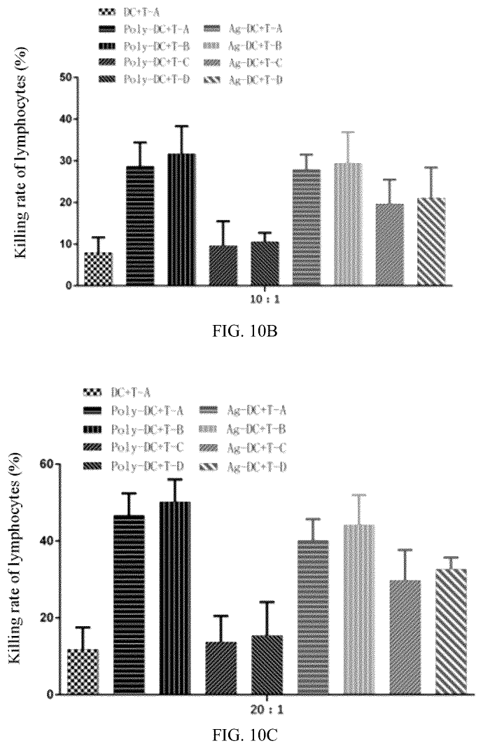

FIGS. 10A-10C show the killing rates of CTLs induced by in vitro stimulation,
where in the Poly-DC+T-A group, CTLs stimulated by the multivalent DC vaccine loaded with a variety of tumor cell lysates (as effector cells) and GC cells of a patient A (as target cells) are co-cultivated in effector-target ratios of 5:1, 10:1, and 20:1. It can be seen that CTLs stimulated by a monovalent vaccine loaded with an autologous GC tumor cell lysate exhibit a significant killing effect for autologous GC cells. The CTLs stimulated by the multivalent vaccine also exhibit a strong tumor-killing ability when encountering autologous EBV-positive tumor cells (patients A and B) but do not exhibit a significant tumor-killing effect when encountering EBV-negative GC cells (patients C and D). The above results show that when there are similar EBV antigens to the multivalent vaccine itself on the surface of autologous tumor cells, the body will be induced to produce a strong immune response. If tumor cells are EBV-negative, the multivalent DC vaccine loaded with EBV antigen information cannot effectively stimulate and activate an anti-tumor immune response in the body, indicating that the Poly-DC multivalent vaccine can effectively inhibit the growth of EBV-positive GC and has a wide application range.

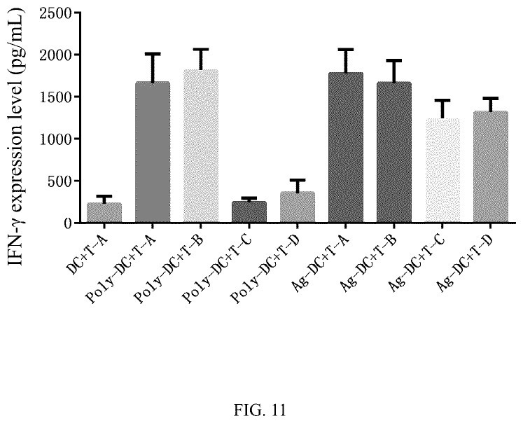

FIG. 11 shows the secretion of IFN-γ,
where it can be seen from the figure that CTLs in the monovalent vaccine group secrete a large amount of IFN-γ when co-cultivated with autologous GC cells. CTLs in the multivalent vaccine group secrete a large amount of IFN-γ when co-cultivated with EBV-positive GC cells derived from different patients but cannot effectively secrete IFN-γ when co-cultivated with EBV-negative NPC cells. There is no significant difference compared with the control group, indicating that the use of the general EBV tumor cell lysate as antigen information can effectively avoid differences in individualized EBV-positive tumor cells and accelerate the industrialization of DC vaccines. The multivalent vaccine loaded with an EBV-positive tumor cell lysate can stimulate and activate an immune system to recognize and kill EBV-positive GC cells and has a strong and extensive immune function-promoting effect.

### DETAILED DESCRIPTION OF THE EMBODIMENTS

The preferred implementations of the present disclosure will be described in detail below in conjunction with examples. It should be understood that the following examples are provided merely for the purpose of illustration and are not intended to limit the scope of the present disclosure. Those skilled in the art can make various modifications and substitutions to the present disclosure without departing from the purpose and spirit of the present disclosure. Unless otherwise specified, the experimental methods used in the following examples are conventional methods. The materials, reagents, and the like used in the following examples are all commercially available unless otherwise specified.

### Example 1: Immunological Study on the Treatment of NPC with a Multivalent DC Vaccine

In this example, tumor cells were collected from each of two EBV-positive NPC patients (denoted as I and II) and two EBV-negative NPC patients (denoted as III and IV) (the patients had signed an informed consent form).

### 1. Isolation of PBMCs from Human Venous Blood

In this example, based on density differences among cell components in peripheral blood, a Ficoll® Paque Plus (GE Healthcare) solution (with a density of 1.075 to 1.089 kg/m³) was added to a peripheral blood sample. Density gradient centrifugation was performed, such that different cell components were separated into different layers so the mononuclear cells could be quickly isolated from human peripheral blood. The peripheral blood mainly includes platelets, mononuclear cells, granulocytes, and red blood cells (RBCs). The platelets have a density of 1.030 to 1.035 kg/m³. The mononuclear cells have a density of 1.075 to 1.090 kg/m³. The granulocytes have a density of 1.092 kg/m³. The RBCs have a density of 1.093 kg/m³.
1) Peripheral blood was collected from a vein of an NPC patient in a centrifuge tube with a corresponding size, and 4.5 mL of a Ficoll® Paque Plus solution was added by a pipette to each of two new centrifuge tubes.
2) A blood sample was drawn by a pipette and slowly added to an upper layer of the Ficoll solution along a wall of the centrifuge tube, 10 mL per centrifuge tube. Then the centrifuge tubes were centrifuged at room temperature and 800 g for 20 min.
3) The centrifuge tubes were taken out. The sample solution in the centrifuge tubes was divided into four layers, including a plasma layer, a mononuclear cell layer, a Ficoll solution layer, an RBC layer, and a granulocyte layer sequentially from top to bottom.
4) The mononuclear cell layer was carefully transferred to a 15 mL centrifuge tube. A PBS/1% FBS solution was added to 14 mL mark. The resulting mixture was pipetted up and down for thorough mixing and then centrifuged at 800 g for 5 min at room temperature.
5) A resulting supernatant was discarded. The bottom of the centrifuge tube was flicked to loosen the cells. Then 14 mL of a PBS/1% FBS solution was added to resuspend the cells. The resulting suspension was pipetted up and down for thorough mixing and then centrifuged at 700 g for 5 min at room temperature.
6) A resulting supernatant was discarded. The bottom of the centrifuge tube was flicked to loosen the cells. Then 14 mL of an RPMI/10% FBS solution was added to resuspend the cells. The resulting suspension was pipetted up and down for thorough mixing and then centrifuged at 400 g for 5 min at room temperature.
7) A resulting supernatant was discarded. The bottom of the centrifuge tube was flicked to loosen the cells. Then 10 mL of an RPMI/10% FBS solution was added to resuspend the cells. The resulting suspension was pipetted up and down for thorough mixing.
8) 10 μL of a resulting cell suspension was transferred to a new 1.5 mL centrifuge tube, and 90 μL of an RPMI/10% FBS solution was added to dilute the cell suspension 10-fold. 10 μL of a diluted cell suspension was taken, 10 μL of Trypan Blue was added for staining, and the resulting mixture was added to a hemacytometer and counted under an inverted microscope.
9) The remaining cell suspension was centrifuged at 700 g for 5 min at room temperature, and a resulting supernatant was discarded. An appropriate amount of PBS/1% FBS was added for subsequent experiments.

### 2. Isolation of DC and T Lymphocytes

2.1 The isolation methods of CD14 mononuclear cells include, but are not limited to, CD14⁺ magnetic bead separation in this example, CD14 negative selection, Miltenyi immunomagnetic cell sorting (MACS), and cell attachment. The isolation principle is based on the specific binding of an antigen and an antibody. A CD14⁺ magnetic bead separation kit can specifically recognize and bind to CD14⁺ cells among PBMCs and is indirectly coupled with magnetic beads through biotin or dextran, such that the CD14⁺ cells can be separated under the action of a high-intensity magnetic field. In this example, the EasySep™ CD14 positive selection kit was used.
1) A PBMC suspension was transferred to a 5 mL FCM tube.
2) An appropriate amount of a selection cocktail solution was added to the FCM tube to a final concentration of 100 μL/mL. The resulting mixture was pipetted up and down for thorough mixing and then incubated at room temperature for 10 min.
3) Magnetic beads were vortexed in a RapidSphere™ solution for 30 s, such that the magnetic beads were dispersed evenly.
4) An appropriate amount of a RapidSphere™ solution was added to the FCM tube to a final concentration of 100 μL/mL. The resulting mixture was pipetted up and down for thorough mixing and then incubated at room temperature for 3 min.
5) An appropriate amount of a PBS/2% FBS with 1 mM EDTA solution was added to the FCM tube to a total volume of 2.5 mL, and a resulting mixture was pipetted up and down for thorough mixing.
6) The FCM tube was vertically inserted into the EasySep™ magnet and incubated for 3 min at room temperature.
7) A magnet was placed invertedly, and a cell solution flowing out from the FCM tube was collected in a 15 mL centrifuge tube, where the magnet was placed invertedly for 3 s. The tube was not shaken. Liquid on the wall of the tube should not be totally removed.
8) The magnet was placed upright and then the FCM tube was taken out.
9) Steps 7 to 10 were repeated twice.
10) 2 mL of RPMI/10% FBS was added to the FCM tube to resuspend cells, and the cells were counted with Trypan Blue.

### 2.2 Experimental Scheme for the Induction of CD14⁺ Mononuclear Cells to Produce iDCs

In vitro, granulocyte-macrophage colony-stimulating factor (GM-CSF) can promote the survival of iDCs and induce the massive proliferation of iDCs. IL-4 can inhibit the overgrowth of macrophages, reduce the expression of CD14 molecule on a cell surface, and induce the differentiation of CD14⁺ mononuclear cells into iDCs.
1) In a clean bench, a CD14⁺ cell suspension was transferred by a pipette to a six-well plate with 2×10⁶ cells/mL in each well. Then 1 μL of human recombinant GM-CSF (20 ng/μL to 200 ng/μL) and 1 μL of human recombinant IL-4 (10 ng/μL to 100 ng/μL) were added to the six-well plate.
2) The six-well plate was placed on a surface of the clean bench, then gently shaken three times back and forth and three times left and right to make the cells dispersed evenly, and incubated in a cell incubator at 37° C. and 5% CO₂ for 3 d.
3) The six-well plate was taken out from the incubator, and then 2 mL of RPMI 1640/10% FBS, 1 μL of human recombinant GM-CSF, and 1 μL of human recombinant IL-4 were added to the six-well plate in a clean bench.
4) The six-well plate was placed on a surface of the clean bench, then gently shaken three times back and forth and three times left and right to make the components dispersed evenly, and incubated in a cell incubator at 37° C. and 5% CO₂ for 2 d.

### 2.3 Loading a Tumor Cell Lysate to Prepare a Multivalent DC Vaccine

1) Construction of an immortalized human B-LCL infected with an EBV strain
a) PBMCs were isolated from peripheral blood and resuspended in 2 mL of an RPMI1640/10% FBS medium.
b) 10 μL of a resulting cell suspension was taken, 90 μL of RPMI/10% FBS was added to dilute 10-fold, and the cells were counted under a microscope.
c) According to a counting result, a required volume of a B95-8 supernatant was calculated, where every 1×10⁶ PBMCs corresponded to 500 μL of the B95-8 supernatant.
d) 10 mL of B95-8 cells were cultivated two days in advance with an initial density of 1×10⁶ cells/mL. The B95-8 cells were cultivated in an incubator for 48 h. Then a resulting cell supernatant was transferred to a centrifuge tube and centrifuged at 2,000 rpm for 15 min, and the remaining B95-8 cells were sterilized and discarded.
e) The B95-8 cell supernatant in the centrifuge tube was filtered through a 0.45 μm filter membrane for later use.
f) PBMCs were collected and centrifuged at 1,000 rpm for 5 min, and a resulting PBMC supernatant was discarded.
g) According to a cell counting result, the PBMCs were resuspended with an appropriate amount of the B95-8 cell supernatant or another EBV suspension (such as GD1, B95-8, M81, HKNPC1 to HKNPC9, SNU-719, and YCCEL1) to obtain a cell suspension in which a concentration of the PBMCs was 1×10⁶/500 μL.
h) A sterile 96-well plate was prepared, and the suspension of PBMCs in B95-8 was added to the 96-well plate at 100 μL/well.
i) The 96-well plate was incubated in a CO₂ incubator for 24 h.
j) The 96-well plate was taken out. Then 100 μL of an R20 medium (RPMI1640/20% FBS, 100 U/mL penicillin and 100 μg/mL streptomycin) was added to each well. The resulting mixture was pipetted up and down for thorough mixing.
k) The 96-well plate was further incubated in an incubator for 6 d, during which a cell status was observed every day to determine whether the cell status underwent the following lymphoblastoid changes: increased cell volume, enriched cytoplasm, spherical shape, aggregated distribution of small colonies, significantly increased cell masses at a bottom of the well, and yellowing medium.
1) After the 6-day cultivation was completed, the medium was changed every 3 d. The upper 100 μL of the medium in each well was carefully removed. Then 100 μL of an R20 medium was added to each well to resuspend the cells in the well. When the medium turned yellow, the medium was changed timely, or the cells were dispensed into new 2 to 4 wells as required. After the number of cells gradually increased, the cells were then combined and transferred to a 24-well plate, a 6-well plate, and a T25 flask.
m) The status of cells after the four-week cultivation was observed under a microscope. An expression level of CD19 on a surface of the cells was analyzed by FCM. The expression level of CD19 on the surface of the immortalized human B-LCL was shown in FIG. 1.
2) Acquisition of tumor cells

A tumor tissue was collected from a patient, immediately rinsed with a medium including 1,000 U/mL penicillin and streptomycin and 2 μg/mL amphotericin for 5 min to 10 min, and then repeatedly rinsed with a serum-free medium. On a clean bench, a vigorously-proliferating tissue at an edge of the tumor tissue was collected, chopped with sharp surgical blades with the mechanical damage minimized, and digested with 0.5% collagenase type IV and 0.5% hyaluronidase at 37° C. for 80 min. The resulting mixture was filtered through a 200-mesh nylon mesh, and the resulting filtrate was centrifuged at 1,000 rpm for 5 min. The resulting cells were resuspended in an RPMI1640 complete medium with 10% FBS, inoculated in a culture plate, and cultivated in a 37° C. and 5% CO₂ incubator. The tumor cells were purified by repeated adherence.

Determination of EBV loads in the blood and tumor tissue of NPC patients: Blood samples were collected from the 4 NPC patients. DNA extraction and PCR were conducted respectively with the MagMAX viral nucleic acid extraction kit (Thermo A42352) and the EBV Real-™ Quant kit (Sacace BioTechnologies Srl, Como, Italy). DNA was extracted from 100 μL of plasma according to a method provided by the manufacturer and eluted with 50 μL of an elution buffer. The NPC tissue cells obtained above were subjected to total DNA extraction according to instructions of the DNeasy Blood & Tissue Kit (Qiagen, Cat. No. 69506) product, and 10 μL of a sample was subjected to EBV quantification by RT-qPCR (EBV Real-™ Quant Kit). In this experiment, a coding region of the EBNA1 gene was selected as an amplification target, and the PCR was conducted according to instructions with a final volume of 25 μL. Primer sequences were as follows:

EBNA1-FP:
5′-CCAGACAGCAGCCAATTGTC-3′, as shown in SEQ ID
NO: 1;
EBNA1-RP:
5′-GGTAGAAGACCCCCTCTTAC-3′, as shown in SEQ ID
NO: 2;
upstream primer for internal reference
β-actin gene:
5′-CTCCATCCTGGCCTCGCTGT-3′, as shown in SEQ ID
NO: 3;
and
downstream primer for internal reference
β-actin gene:
5′-GCTGTCACCTTCACCGTTCC-3′, as shown in SEQ ID
NO: 4.

Copy numbers of EBV-DNA in the blood and tumor tissue of the 4 patients were compared, and results were shown in FIGS. 2A-2B.

The repeated freezing-thawing method is a common mechanical lysis method, which usually consists of freezing and thawing. A principle of the method is as follows: The generation of intracellular ice particles and the increase of salt concentration of the remaining cell solution cause swelling, such that the cell structure is broken and cells die, but the immunogenicity of the cells is retained. The freezing is usually conducted in liquid nitrogen or on ice at −20° C., and the thawing can be conducted through heat shock in a water bath at 37° C., 50° C., 65° C., or 100° C., which is milder than the chemical lysis.
a) A temperature of a water bath was pre-set to 37° C.
b) A culture of each of the C666-1 NPC cell line, the immortalized human B-LCLs constructed by different virus strains, and the tumor cells of 4 NPC patients were collected (at least 3×10⁷ cells) and centrifuged at 700 g for 5 min at room temperature.
c) A resulting supernatant was discarded, and resulting cells were resuspended in RPMI/10% FBS to obtain a cell suspension.
d) The cells were counted with trypan blue.
e) The cell suspension was centrifuged at 700 g for 5 min at room temperature, and a resulting supernatant was carefully removed.
f) Resulting cells were resuspended with RPMI/10%FBS in a 1 mL freezing tube with a density of 5×10⁶/mL.
g) The cells were frozen in liquid nitrogen for 20 s.
h) The cells were immediately thawed quickly and completely in a 37° C. water bath.
i) Steps g) and h) were repeated 4 times (5 times in total).
j) A tumor cell lysate was stored in liquid nitrogen before use.
3) Acquisition of a DC vaccine
a) Cytokines GM-CSF and IL-4 were used to make mononuclear cells derived from blood differentiated into iDCs, after cultivated with GM-CSF and IL-4 for 5 days, the mononuclear cells differentiated into iDCs:
monovalent DC vaccine: 5×10⁶ iDCs were co-cultivated with a lysate of 2.5×10⁷ autologous tumor cells for at least 2 h. Then cytokines such as TNF-α were added to stimulate the maturation of DCs to obtain the monovalent DC vaccine.
multivalent DC vaccine: 5×10⁶ DCs were co-cultivated with each of different tumor cell lysates, such as a lysate of 2.5×10⁷ C666-1 tumor cells or an M81-LCL tumor cell lysate, for at least 2 h. Then cytokines, such as TNF-α, were added to stimulate the maturation of DCs, and multiple types of DCs loaded with tumor cell antigen information were mixed in equal amounts in a DC medium to obtain the multivalent DC vaccine loaded with tumor cell lysate antigens.

Surface molecular markers on iDCs and mDCs were detected by FCM, such as CD11c, CD14, CD40, CD80, CD83, CD86, HLA-DR, and HLA-ABC. A morphological image of mDCs is shown in FIG. 3, and FCM results of the expression of the surface marker molecules on DCs are shown in FIGS. 4A-4H.
b) DCs in each DC vaccine were derived from a patient, and the following 4 vaccines were prepared:

TABLE 1

| | Composition | | | | | | | | |
|---|---|---|---|---|---|---|---|---|---|
| | Ag- | DC- | DC- | DC- | DC- | Ag- | Ag- | Ag- | Ag- |
| Group | Poly | I | II | III | IV | I | II | III | IV |
| Poly- | + | + | | | | | | | |
| DC-I | | | | | | | | | |
| Poly- | + | | + | | | | | | |
| DC-II | | | | | | | | | |
| Poly- | + | | | + | | | | | |
| DC-III | | | | | | | | | |
| Poly- | + | | | | + | | | | |
| DC-IV | | | | | | | | | |
| Ag-DC-I | | + | | | | + | | | |
| Ag-DC-II | | | + | | | | + | | |
| Ag- | | | | + | | | | + | |
| DC-III | | | | | | | | | |
| Ag- | | | | | + | | | | + |
| DC-IV | | | | | | | | | |

* Ag-Poly indicates that it carries different tumor cell lysate antigen information, such as a C666-1 tumor cell lysate or tumor cell lysates of B95-8 and M81-LCLs. DC-I represents autologous DCs from the EBV-positive NPC patient I. DC-II represents autologous DCs from the EBV-positive NPC patient II. DC-III represents autologous DCs from the EBV-negative NPC patient III. DC-IV represents autologous DCs from the EBV-negative NPC patient IV. Ag-I represents tumor cell lysate antigen information of the NPC patient I, Ag-II represents tumor cell lysate antigen information of the NPC patient II, Ag-III represents tumor cell lysate antigen information of the NPC patient III, and Ag-IV represents tumor cell lysate antigen information of the NPC patient IV. Poly-DC-I represents a multivalent DC vaccine for patient I, Ag-DC-I represents a monovalent DC vaccine for patient I that is loaded with tumor cell lysate information of the patient I, and so on.

### 2.4 Preparation of T Lymphocytes

1) PBMCs isolated from the same person were cultivated in a 37° C. and 5% CO₂ incubator for 2 h, and then suspended cells were collected and prepared into 1 mL of a cell suspension.
2) The cell suspension was added to a nylon wool fiber column incubated at 37° C. The column was laid flat. Then 200 μL of pre-warmed 10% FBS-containing RPMI 1640 was added for sealing, and then the column was incubated at 37° C. for 2 h.
3) The nylon wool fiber column was subjected to elution with 10% FBS RPMI 1640 at a flow rate of about 1 mL/min, and 10 mL of a cell suspension was collected at the beginning, which was rich in T cells and NK cells.
4) The cell suspension was centrifuged at 700 g for 5 min at room temperature. A cell pellet was collected, counted, and adjusted to a cell concentration of 1×10⁷ cells/mL and placed in an 80 IU/mL IL-2-containing RPMI1640 complete medium for later use.

Alternatively, the magnetic bead separation method can be used, that is, T lymphocytes can be isolated through CD3⁺ magnetic beads. Cells were first incubated with an anti-surface antigen monoclonal antibody (mAb) for 12 min (50 μL of an anti-CD3 mAb was used for every 10⁷ cells), then washed and incubated with 100 μL of a biotin-labeled goat anti-mouse secondary antibody for 10 min, then washed and incubated with 25 μL of FITC-labeled streptavidin for 8 min, and then washed and incubated with biotin-labeled magnetic beads (100 μL of magnetic beads was added when the anti-CD3 mAb was added) for 8 min. After the above reactions were completed, 1 mL of 1% BSA-containing PBS was added for washing, and a resulting mixture was centrifuged at 2,000 r/min for 10 min. T lymphocytes were isolated through immunomagnetic separation of a magnetic cell separator (MACS).

### 3. Acquisition of CTLs Induced by In Vitro Stimulation

A multivalent DC vaccine and normal mDCs were each resuspended in an RPMI complete medium with a cell density adjusted to 2×10⁵ cells/mL. The isolated autologous T lymphocyte suspension was adjusted with an RPMI complete medium to a cell density of 1.6×10⁶/mL. The following experimental groups were set, and 1 mL of a corresponding DC vaccine and T lymphocytes were added in each group, as shown in Table 2:

TABLE 2

| | | | | | | | | | |
|---|---|---|---|---|---|---|---|---|---|
| DC-I | + | | | | | | | | |
| Poly-DC-I | | + | | | | | | | |
| Poly-DC-II | | | + | | | | | | |
| Poly-DC-III | | | | + | | | | | |
| Poly-DC-IV | | | | | + | | | | |
| Ag-DC-I | | | | | | + | | | |
| Ag-DC-II | | | | | | | + | | |
| Ag-DC-III | | | | | | | | + | |
| Ag-DC-IV | | | | | | | | | + |
| T-I | + | + | | | | + | | | |
| T-II | | | + | | | | + | | |
| T-III | | | | + | | | | + | |
| T-IV | | | | | + | | | | + |
| Group | a | b | c | d | e | f | g | h | i |

* DC-I represents DCs derived from the patient I. Poly-DC-I represents a multivalent DC vaccine for patient I, Poly-DC-II represents a multivalent DC vaccine for patient II, Poly-DC-III represents a multivalent DC vaccine for patient III, and Poly-DC-IV represents a multivalent DC vaccine for patient IV. Ag-DC-I represents a monovalent DC vaccine for patient I that is loaded with tumor cell lysate antigen of the patient I, Ag-DC-II represents a monovalent DC vaccine for patient II that is loaded with tumor cell lysate antigen of the patient II, Ag-DC-III represents a monovalent DC vaccine for patient III that is loaded with tumor cell lysate antigen of the patient III, and Ag-DC-IV represents a monovalent DC vaccine for patient IV that is loaded with tumor cell lysate antigen of the patient IV. T-I represents T lymphocytes derived from patient I, T-II represents T lymphocytes derived from patient II, T-III represents T lymphocytes derived from patient III, and T-IV represents T lymphocytes derived from patient IV. Group a represents an co-culture of DC-I and T-I, Group b represnets an co-culture of Poly-DC-I and T-I, Group c represnets an co-culture of Poly-DC-II and T-II, Group d represnets an co-culture of Poly-DC-III and T-III, Group e represnets an co-culture of Poly-DC-IV and T-IV, Group f represnets an co-culture of Ag-DC-I and T-I, Group g represnets an co-culture of Ag-DC-II and T-II, Group h represnets an co-culture of Ag-DC-III and T-III, and Group i represnets an co-culture of Ag-DC-IV and T-IV.

The same helper cytokines were added in each of the above experimental groups with an IL-2 content of 1,000 U/mL, an IL-12 content of 1,500 U/mL, a Poly(I:C) content of 10 mg/mL, and a TNF-α content of 1,000 U/mL. Cells were cultivated for 2 weeks in a 37° C. and 5% CO₂ constant-temperature and constant-humidity incubator. IL-2 was added with a final concentration of 30 U/mL. Then the corresponding DC vaccines (2×10⁵ cells in each group) were added for secondary stimulation, and the cells were further cultivated for one week and harvested on day 21.

### 4. Assay of Killing Activities of Collected Cells on NPC Cells in Different Patients

Some of the cells collected above were centrifuged and resuspended in an RPMI1640 complete medium. A cell concentration was adjusted, and the cells were added as effector cells to a 96-well culture plate at 4×10⁵/well, 2×10⁵/well, and 1×10⁵/well to set three experimental groups with different effector-target ratios. Tumor cells from different NPC patients were adopted as target cells. 2×10⁴ tumor cells of an NPC patient were added as target cells to each well with a final volume of 200 μL. The set of experimental groups are shown in Table 3. A control group without lymphocytes and a blank medium control group without cells were set, and 5 replicate wells were also set for each of the control groups. 24 h later, free effector cells in each well were removed, the plate was washed twice with PBS, 100 μL of a reagent including 20 μL of CCK8 was added to each well, and the cells were further cultivated for 2 h. The absorbance (OD) at 450 nm was determined by a microplate reader, and a killing rate (%) of specific lymphocytes was calculated. The killing rates of CTLs induced by in vitro stimulation were shown in FIGS. 5A-5C.

TABLE 3

| | Effector cell | | | | | | | | |
|---|---|---|---|---|---|---|---|---|---|
| | Group | Group | Group | Group | Group | Group | Group | Group | Group |
| Target cell | a | b | c | d | e | f | g | h | i |
| Tumor cells of patient I | + | + | | | | + | | | |
| Tumor cells of patient II | | | + | | | | + | | |
| Tumor cells of patient III | | | | + | | | | + | |
| Tumor cells of patient IV | | | | | + | | | | + |
| Group | a-I | b-I | c-II | d-III | e-IV | f-I | g-II | h-III | i-IV |

* Activated T cells from Groups a-i of Table 2 were used to conduct killing experiments on autologous tumor cells derived from patients I-IV, respectively. Group a-I represents an co-culture of tumor cells of patient I and CTL cells of group a in Table 2, Group b-I represents an co-culture of tumor cells of patient I and CTL cells of group b in Table 2, Group c-II represents an co-culture of tumor cells of patient II and CTL cells of group c in Table 2, Group d-III represents an co-culture of tumor cells of patient III and CTL cells of group d in Table 2, Group e-IV represents an co-culture of tumor cells of patient IV and CTL cells of group e in Table 2, Group f-I represents an co-culture of tumor cells of patient I and CTL cells of group f in Table 2, Group g-II represents an co-culture of tumor cells of patient II and CTL cells of group g in Table 2, Group h-III represents an co-culture of tumor cells of patient III and CTL cells of group h in Table 2, and Group i-IV represents an co-culture of tumor cells of patient IV and CTL cells of group i in Table 2. Among them, Group a-I indicate a killing effect of DC vaccine without antigen loading, Groups b-I, c-II, d-III, and e-IV indicate killing effects of multivalent DC vaccine, and Groups f-I, g-II, h-III, and i-IV indicate killing effects of monovalent DC vaccine.

### 5. In Vitro Detection of Secretion of IFN-γ

The CTL effector cells of each of the above groups and the tumor cells of each of the 4 NPC patients were mixed in a U-bottom 96-well plate according to an effector-target ratio of 20:1 and cultivated for 72 h. A content of IFN-γ in a culture supernatant was detected with an IFN-γ ELISA kit according to instructions, and the results are shown in FIG. 6.

### Example 2: Immunological Study on the Resistance of a Multivalent DC Vaccine to EBV-Positive GC

In this example, two EBV-positive GC patients (A and B) and two EBV-negative GC patients (C and D) who signed an informed consent form were selected.

### 1. Isolation of PBMCs from Human Venous Blood

In this example, based on density differences among cell components in peripheral blood, a Ficoll® Paque Plus (GE Healthcare) solution (with a density of 1.075 to 1.089 kg/m³) was added to a peripheral blood sample, and then the density gradient centrifugation was conducted, such that different cell components were separated into different layers and thus the mononuclear cells could be quickly isolated from human peripheral blood. The peripheral blood mainly includes platelets, mononuclear cells, granulocytes, and RBCs, where the platelets have a density of 1.030 to 1.035 kg/m³, the mononuclear cells have a density of 1.075 to 1.090 kg/m³, the granulocytes have a density of 1.092 kg/m³, and the RBCs have a density of 1.093 kg/m³.
1) Peripheral blood was collected from a vein of a GC patient in a centrifuge tube with a corresponding size, and 4.5 mL of a Ficoll® Paque Plus solution was added by a pipette to each of two new centrifuge tubes.
2) A blood sample was drawn by a pipette and slowly added to an upper layer of the Ficoll solution along a wall of the centrifuge tube, 10 mL per centrifuge tube; then the centrifuge tubes were centrifuged at 800 g for 20 min at room temperature.
3) The centrifuge tubes were taken out. The sample solution in the centrifuge tubes was divided into four layers, including a plasma layer, a mononuclear cell layer, a Ficoll solution layer, an RBC layer, and a granulocyte layer sequentially from top to bottom.
4) The mononuclear cell layer was carefully transferred to a 15 mL centrifuge tube, a PBS/1% FBS solution was added to 14 mL, and a resulting mixture was pipetted up and down for thorough mixing and then centrifuged at 800 g for 5 min at room temperature.
5) The resulting supernatant was discarded. The bottom of the centrifuge tube was flicked to loosen the cells. Then 14 mL of a PBS/1% FBS solution was added to resuspend the cells. The resulting suspension was pipetted up and down for thorough mixing and then centrifuged at room temperature at 700 g for 5 min.
6) A resulting supernatant was discarded. The bottom of the centrifuge tube was flicked to loosen the cells. Then 14 mL of an RPMI/10% FBS solution was added to resuspend the cells. The resulting suspension was pipetted up and down for thorough mixing and then centrifuged at room temperature and 400 g for 5 min.
7) The resulting supernatant was discarded. The bottom of the centrifuge tube was flicked to loosen the cells. Then 10 mL of an RPMI/10% FBS solution was added to resuspend the cells, and a resulting suspension was pipetted up and down for thorough mixing.
8) 10 μL of a resulting cell suspension was transferred to a new 1.5 mL centrifuge tube, and 90 μL of an RPMI/10% FBS solution was added to dilute the cell suspension 10-fold. 10 μL of a diluted cell suspension was taken, 10 μL of Trypan Blue was added for staining, and a resulting mixture was added to a hemacytometer and counted under an inverted microscope.
9) The remaining cell suspension was centrifuged at room temperature at 700 g for 5 min. The resulting supernatant was discarded, and an appropriate amount of PBS/1% FBS was added for subsequent experiments.

### 2. Experimental Scheme for the Induction of CD14⁺ Mononuclear Cells to Produce iDCs

In vitro, GM-CSF can promote the survival of iDCs and induce the massive proliferation of iDCs. IL-4 can inhibit the overgrowth of macrophages, reduce the expression of CD14 molecule on a cell surface, and induce the differentiation of CD14⁺ mononuclear cells into iDCs.
1) In a clean bench, a CD14⁺ cell suspension was transferred by a pipette to a six-well plate with 2×10⁶ cells/mL in each well. Then 1 μL of human recombinant GM-CSF (20 ng/μL to 200 ng/μL) and 1 μL of human recombinant IL-4 (10 ng/μL to 100 ng/μL) were added to the six-well plate.
2) The six-well plate was placed on a surface of the clean bench, then gently shaken three times back and forth and three times left and right to make the cells dispersed evenly, and incubated in a cell incubator at 37° C. and 5% CO₂ for 3 d.
3) The six-well plate was taken out from the incubator. Then 2 mL of RPMI 1640/10% FBS, 1 μL of human recombinant GM-CSF, and 1 μL of human recombinant IL-4 were added to the six-well plate in a clean bench.
4) The six-well plate was placed on a surface of the clean bench, then gently shaken three times back and forth and three times left and right to make the components dispersed evenly, and incubated in a cell incubator at 37° C. and 5% CO₂ for 2 d. Some iDCs were collected, and the expression of costimulatory factors on the cell surface thereof was detected by FCM. Detection results are shown in FIGS. 7A-7B.

### 3. A Method for Treating a Tumor Tissue of an EBV-Positive GC Patient and a Method for Preparing a Tumor Cell Lysate were the Same as Above

A tumor tissue was collected from a patient, immediately rinsed with a medium including 1,000 U/mL penicillin and streptomycin and 2 μg/mL amphotericin for 5 min to 10 min, and then repeatedly rinsed with a serum-free medium. On a clean bench, a vigorously-proliferating tissue at an edge of the tumor tissue was collected, chopped with sharp surgical blades with the mechanical damage minimized, and digested with 0.5% collagenase type IV and 0.5% hyaluronidase at 37° C. for 80 min. The resulting mixture was filtered through a 200-mesh nylon mesh, and the resulting filtrate was centrifuged at 1,000 rpm for 5 min. Resulting cells were resuspended in an RPMI1640 complete medium with 10% FBS, inoculated into a culture plate, and cultivated in a 37° C. and 5% CO₂ incubator. The tumor cells were purified by repeated adherence combined with mechanical scraping.

Determination of EBV loads in the blood and GC tissue samples of GC patients: Blood samples were collected from the 4 GC patients. DNA extraction and PCR were conducted respectively with the MagMAX viral nucleic acid extraction kit (Thermo A42352) and the EBV Real-™ Quant kit (Sacace BioTechnologies Srl, Como, Italy). DNA was extracted from 100 μL of plasma according to a method provided by the manufacturer and eluted with 50 μL of an elution buffer. The GC tissue cells obtained above were subjected to total DNA extraction according to instructions of the DNeasy Blood & Tissue Kit (Qiagen, Cat. No. 69506) product, and 10 μL of a sample was subjected to EBV quantification by RT-qPCR (EBV Real-™ Quant Kit). In this experiment, a coding region of the EBNA1 gene was selected as an amplification target, and the PCR was conducted according to instructions with a final volume of 25 μL.

Sequences of the primers were as follows:

EBNA1-FP:
5′-CCAGACAGCAGCCAATTGTC-3′, as shown in SEQ ID
NO: 1;
EBNA1-RP:
5′-GGTAGAAGACCCCCTCTTAC-3′, as shown in SEQ ID
NO: 2;
upstream primer for internal reference
β-actin gene:
5′-CTCCATCCTGGCCTCGCTGT-3′, as shown in SEQ ID
NO: 3;
and
downstream primer for internal reference
β-actin gene:
5′-GCTGTCACCTTCACCGTTCC-3′, as shown in SEQ ID
NO: 4.

Copy numbers of EBV-DNA in the plasma and tumor tissue of the 4 patients were compared, and results were shown in FIGS. 8A-8B.

### 4. Preparation of a DC Vaccine for EBV-Positive GC

The cytokines GM-CSF and IL-4 were used to make mononuclear cells in blood differentiated into iDCs. The mononuclear cells were cultivated with GM-CSF and IL-4 for 5 d to differentiate into iDCs. A lysate of LCLs constructed from EBV-associated GC virus strains (such as SNU719-LCLs and YCCEL1-LCLs) or a lysate of a GC tumor cell line (such as GT38 and PT) was added to allow an cultivation for 2 h, and then cytokines, such as TNF-α, were added to stimulate the maturation of DCs.

Monovalent DC vaccine: 5×10⁶ DCs were co-cultivated with a lysate of 2.5×10⁷ autologous tumor cells for at least 2 h, and then cytokines, such as TNF-α, were added to stimulate the maturation of DCs to obtain the monovalent DC vaccine.

Multivalent DC vaccine: 5×10⁶ DCs were co-cultivated with each of different cell lysates, such as a lysate of 2.5×10⁷ YCCEL1-LCL or GCEBV-LCL tumor cells or a lysate of a GT38 or PT EBV-positive GC cell line, for at least 2 h. Then cytokines, such as TNF-α, were added to stimulate the maturation of DCs. Multiple types of DCs loaded with tumor cell antigen information were mixed in equal amounts in a DC medium to obtain the multivalent DC vaccine loaded with tumor cell lysate antigens.

The following 4 vaccines were prepared according to Table 4, and culture supernatants of the normally-cultivated DCs and the vaccine groups were each collected to detect the expression of IL12. Results were shown in FIG. 9.

TABLE 4

| | Composition | | | | | | | | |
|---|---|---|---|---|---|---|---|---|---|
| | Ag- | | | | | | | | |
| Group | Poly | DC-A | DC-B | DC-C | DC-D | Ag-A | Ag-B | Ag-C | Ag-D |
| Poly-DC-A | + | + | | | | | | | |
| Poly-DC-B | + | | + | | | | | | |
| Poly-DC-C | + | | | + | | | | | |
| Poly-DC-D | + | | | | + | | | | |
| Ag-DC-A | | + | | | | + | | | |
| Ag-DC-B | | | + | | | | + | | |
| Ag-DC-C | | | | + | | | | + | |
| Ag-DC-D | | | | | + | | | | + |

* Ag-Poly indicates that it carries antigen information of various tumor cell lysates, such as tumor cell lysates of YCCEL1-LCLs and GCEBV-LCLs or EBV-positive GC cell lines, such as GT38 and PT. DC-A represents DCs of patient A, DC-B represents DCs of patient B, DC-C represents DCs of patient C, and DC-D represents DCs of patient D. Ag-A represents tumor cell lysate antigen information of GC patient A, Ag-B represents tumor cell lysate antigen information of GC patient B, Ag-C represents tumor cell lysate antigen information of GC patient C, and Ag-D represents tumor cell lysate antigen information of GC patient D. Poly-DC-A represents a multivalent DC vaccine for patient A. Ag-DC-A represents a monovalent DC vaccine for the patient A that is loaded with tumor cell lysate information of the patient A, and so on.

### 5. Preparation of T Lymphocytes

The magnetic bead separation method can be used, that is, T lymphocytes can be isolated through CD3⁺ magnetic beads. Cells were first incubated with an anti-surface antigen mAb for 12 min (50 μL of an anti-CD3 mAb was used for every 10⁷ cells), then washed and incubated with 100 μL of a biotin-labeled goat anti-mouse secondary antibody for 10 min, then washed and incubated with 25 μL of FITC-labeled streptavidin for 8 min, and then washed and incubated with biotin-labeled magnetic beads (100 μL of magnetic beads was added when the anti-CD3 mAb was added) for 8 min. After the above reactions were completed, 1 mL of 1% BSA-containing PBS was added for washing, and a resulting mixture was centrifuged at 2,000 r/min for 10 min. T lymphocytes were isolated through immunomagnetic separation of a magnetic cell separator (MACS).

### 6. Acquisition of CTLs Induced by In Vitro Stimulation

A multivalent DC vaccine, a monovalent DC vaccine, and normal mDCs were each resuspended in an RPMI complete medium with a cell density adjusted to 2×10⁵ cells/mL. The isolated autologous T lymphocyte suspension was adjusted with an RPMI complete medium to a cell density of 1.6×10⁶/mL. The following experimental groups were set, and 1 mL of a corresponding DC vaccine and T lymphocytes were added in each group, as shown in Table 5:

TABLE 5

| | | | | | | | | | |
|---|---|---|---|---|---|---|---|---|---|
| DC-A | + | | | | | | | | |
| Poly-DC-A | | + | | | | | | | |
| Poly-DC-B | | | + | | | | | | |
| Poly-DC-C | | | | + | | | | | |
| Poly-DC-D | | | | | + | | | | |
| Ag-DC-A | | | | | | + | | | |
| Ag-DC-B | | | | | | | + | | |
| Ag-DC-C | | | | | | | | + | |
| Ag-DC-D | | | | | | | | | + |
| T-A | + | + | | | | + | | | |
| T-B | | | + | | | | + | | |
| T-C | | | | + | | | | + | |
| T-D | | | | | + | | | | + |
| Group | a | b | c | d | e | f | g | h | i |

*DC-A represents DC derived from patient A. Poly-DC-A represents multivalent DC vaccine suitable for patient A, Poly-DC-B represents multivalent DC vaccine suitable for patient B, Poly-DC-C represents multivalent DC vaccine suitable for patient C, and Poly-DC-D represents multivalent DC vaccine suitable for patient D. Ag-DC-A represents monovalent DC vaccine of patient A that is loaded with tumor cell lysate antigen of the patient A, Ag-DC-B represents monovalent DC vaccine of patient B that is loaded with tumor cell lysate antigen of the patient B, Ag-DC-C represents monovalent DC vaccine of patient C that is loaded with tumor cell lysate antigen of the patient C, and Ag-DC-D represents monovalent DC vaccine of patient D that is loaded with tumor cell lysate antigen of the patient D. T-A represents T lymphocytes derived from patient A, T-B represents T lymphocytes derived from patient B, T-C represents T lymphocytes derived from patient C, and T-D represents T lymphocytes derived from patient D. Group a represents an co-culture of DC-A and T-A, Group b represents an co-culture of of Poly-DC-A and T-A, Group c represents an co-culture of Poly-DC-B and T-B, Group d represents an co-culture of Poly-DC-C and T-C, Group e represents an co-culture of Poly-DC-D and T-D, Group f represents an co-culture of Ag-DC-A and T-A, Group g represents an co-culture of Ag-DC-B and T-B, Group h represents an co-culture of Ag-DC-C and T-C, and Group i represents an co-culture of Ag-DC-D and T-D.

The same helper cytokines were added in each of the above experimental groups with an IL-2 content of 1,000 U/mL, an IL-12 content of 1,500 U/mL, a Poly(I:C) content of 10 mg/mL, and a TNF-α content of 1,000 U/mL. Cells were cultivated for 2 weeks in a 37° C. and 5% CO₂ constant-temperature and constant-humidity incubator. IL-2 was added with a final concentration of 30 U/mL. Then the corresponding DC vaccines (2×10⁵ cells in each group) were added for secondary stimulation, and the cells were further cultivated for one week and harvested on day 21. Then the immunological function of the multivalent vaccine was detected.

### 7. Assay of Killing Activities of Collected CTLs on GC Cells in Different Patients

Some of the cells collected above were centrifuged and resuspended in an RPMI1640 complete medium. A cell concentration was adjusted, and the cells were added as effector cells to a 96-well culture plate at 4×10⁵/well, 2×10⁵/well, and 1×10⁵/well to set three experimental groups with different effector-target ratios. Tumor cells from different EBV-positive GC patients were adopted as target cells. 2×10⁴ tumor cells of a GC patient were added as target cells to each well with a final volume of 200 μL. The set of experimental groups are shown in Table 6. A control group without lymphocytes and a blank medium control group without cells were set, and 5 replicate wells were also set for each of the control groups. 24 h later, free effector cells in each well were removed, the plate was washed twice with PBS, 100 μL of a reagent including 20 μL of CCK8 was added to each well, and the cells were further cultivated for 2 h. The absorbance (OD) at 450 nm was determined by a microplate reader, and a killing rate (%) of specific lymphocytes was calculated. The killing rates of CTLs induced by in vitro stimulation were shown in FIGS. 10A-10C.

TABLE 6

| | Effector cell | | | | | | | | |
|---|---|---|---|---|---|---|---|---|---|
| Target cell | a | b | c | d | e | f | g | h | i |
| Tumor cells of | + | + | | | | + | | | |
| patient A | | | | | | | | | |
| Tumor cells of | | | + | | | | + | | |
| patient B | | | | | | | | | |
| Tumor cells of | | | | + | | | | + | |
| patient C | | | | | | | | | |
| Tumor cells of | | | | | + | | | | + |
| patient D | | | | | | | | | |
| Group | DC + T- | Poly- | Poly- | Poly- | Poly- | Ag- | Ag- | Ag- | Ag- |
| | A | DC + T- | DC + T- | DC + T- | DC + T- | DC + T- | DC + T- | DC + T- | DC + T- |
| | | A | B | C | D | A | B | C | D |

* Activated T cells from Groups a-i of Table 5 were used to conduct killing experiments on autologous tumor cells derived from patients A-D, respectively. Group DC + T-A represents an co-culture of tumor cells of patient A and CTL cells of group a in Table 5, Group Poly-DC + T-A represents an co-culture of tumor cells of patient A and CTL cells of group b in Table 5, Group Poly-DC + T-B represents an co-culture of tumor cells of patient B and CTL cells of group c in Table 5, Group Poly-DC + T-C represents an co-culture of tumor cells of patient C and CTL cells of group d in Table 5, Group Poly-DC + T-D represents an co-culture of tumor cells of patient D and CTL cells of group e in Table 5, Group Ag-DC + T-A represents an co-culture of tumor cells of patient A and CTL cells of group f in Table 5, Group Ag-DC + T-B represents an co-culture of tumor cells of patient B and CTL cells of group g in Table 5, Group Ag-DC + T-C represents an co-culture of tumor cells of patient C and CTL cells of group h in Table 5, and Group Ag-DC + T-D represents an co-culture of tumor cells of patient D and CTL cells of group i in Table 5. Among them, Group DC + T-A indicate a killing effect of DC vaccine without antigen loading, Groups Poly-DC + T-A, Poly-DC + T-B, Poly-DC + T-C, and Poly-DC + T-D indicate killing effects of multivalent DC vaccine, and Groups Ag-DC + T-A, Ag-DC + T-B, Ag-DC + T-C, and Ag-DC + T-D indicate killing effects of monovalent DC vaccine.

### 8. In Vitro Detection of Secretion of IFN-γ

The CTL effector cells of each of the above groups and the tumor cells of each of the 4 different patients were mixed in a U-bottom 96-well plate according to an effector-target ratio of 20:1 and cultivated for 72 h. A content of IFN-γ in a culture supernatant was detected with an IFN-γ ELISA kit according to instructions, and the results are shown in FIG. 11.

What is claimed is:

1. A tumor complex antigen, comprising a tumor cell lysate of human immortalized B lymphoblastoid cell lines (B-LCLs) derived from different Epstein-Barr virus (EBV) strains and/or an EBV-positive tumor cell lysate, wherein the tumor cell lysate of the human immortalized B-LCLs derived from different EBV strains is at least one selected from the group consisting of GD1, B95-8, M81, HKNPC1 to HKNPC9, SNU-719, and YCCEL1; and the EBV-positive tumor cell lysate is at least one selected from the group consisting of C666-1, HNE1, and CCL85.

2. A multivalent dendritic cell (DC) vaccine, wherein the multivalent DC vaccine carries the tumor complex antigen according to claim 1; and the multivalent DC vaccine carrying the tumor complex antigen is loaded with at least one EBV-associated tumor cell lysate or at least one lymphoblastoid cell line (LCL) tumor cell lysate.

3. The multivalent DC vaccine according to claim 2, wherein the tumor cell lysate of the human immortalized B-LCLs derived from different EBV strains is a tumor cell lysate of one or more selected from the group consisting of human immortalized B-LCLs resulting from a transformation by EBVs of GD1, B95-8, M81, HKNPC1 to HKNPC9, SNU-719, and YCCEL1; and the EBV-positive tumor cell lysate is C666-1, HNE1, or CCL85.

4. The multivalent DC vaccine according to claim 2, wherein the multivalent DC vaccine comprises a first adjuvant or a cytokine for an adjuvant therapy.

5. (canceled)

6. The multivalent DC vaccine according to claim 4, wherein the first adjuvant is one selected from the group consisting of PloyI:C, LPS, and OK432; and the cytokine for the adjuvant therapy is TNF-α or IL-12.

7. The multivalent DC vaccine according to claim 2, wherein each of the tumor cell lysates is specifically used at an amount of 2.5×10⁷ to 2.5×10⁹ cells.

8. A method of use of the tumor complex antigen according to claim 1 in a preparation of a drug for preventing or treating an EBV-associated tumor.

9. The method according to claim 8, wherein the EBV-associated tumor comprises EBV-associated gastric carcinoma (GC), EBV-positive lymphoma, and nasopharyngeal carcinoma (NPC).

10. The method according to claim 9, wherein the drug comprises a multivalent DC vaccine, the multivalent DC vaccine carries the tumor complex antigen; and the multivalent DC vaccine carrying the tumor complex antigen is loaded with at least one EBV-associated tumor cell lysate or at least one lymphoblastoid cell line (LCL) tumor cell lysate.

11. The multivalent DC vaccine according to claim 3, wherein the multivalent DC vaccine comprises a first adjuvant or a cytokine for an adjuvant therapy.

12. The multivalent DC vaccine according to claim 3, wherein each of the tumor cell lysates is specifically used at an amount of 2.5×10⁷ to 2.5×10⁹ cells.

13. The multivalent DC vaccine according to claim 6, wherein each of the tumor cell lysates is specifically used at an amount of 2.5×10⁷ to 2.5×10⁹ cells.

14. The method according to claim 10, wherein the tumor cell lysate of the human immortalized B-LCLs derived from different EBV strains is a tumor cell lysate of one or more selected from the group consisting of human immortalized B-LCLs resulting from a transformation by EBVs of GD1, B95-8, M81, HKNPC1 to HKNPC9, SNU-719, and YCCEL1; and the EBV-positive tumor cell lysate is C666-1, HNE1, or CCL85.

15. The method according to claim 10, wherein the multivalent DC vaccine comprises a first adjuvant or a cytokine for an adjuvant therapy.

16. The method according to claim 15, wherein the first adjuvant is one selected from the group consisting of PloyI:C, LPS, and OK432; and the cytokine for the adjuvant therapy is TNF-α or IL-12.

17. The method according to claim 10, wherein each of the tumor cell lysates is specifically used at an amount of 2.5×10⁷ to 2.5×10⁹ cells.
